(19) United States
(12) Patent Application Publication
(10) Pub. No.: US 2017/0348496 A1
(43) Pub. Date: Dec. 7, 2017

(54) Can and Actuator Assembly

(71) Applicant: Norton (Waterford) Limited, Waterford (IE)

(72) Inventor: Sean Leane, Waterford (IE)

(73) Assignee: Norton (Waterford) Limited, Waterford (IE)

(21) Appl. No.: 15/536,852

(22) PCT Filed: Dec. 17, 2014
(86) PCT No.: PCT/EP2015/080214

§ 371 Date: Jun. 16, 2017

(30) Foreign Application Priority Data
Dec. 17, 2014 (GB) 1422496.8

(51) Int. Cl. A61M 15/00; G01L 5/00; G01L 1/16; A61M 11/00

(52) U.S. Cl. CPC A61M 15/009; G01L 1/16; G01L 5/0076; A61M 15/0021; A61M 11/00; A61M 2205/332; A61M 2207/10

(57) ABSTRACT

An apparatus for inserting a canister (3) into an inhaler actuator device (20), wherein the apparatus comprises a force sensor (25) adapted to measure a reaction force between the canister and actuator device as the canister moves relative to the actuator device, and a corresponding method.

### BACKGROUND

The present invention is concerned with an apparatus and method for assembling a metered dose inhaler. Specifically the invention is concerned with assembling an aerosol canister (which has been filled with a medicament) into an inhaler device.

Metered Dose Inhalers (MDIs) are commonly used by patients to administer medicaments into the lungs through inhalation by the patient. A typical condition where MDIs are used is in the treatment of asthma.

MDIs commonly comprise two components: an actuator device and an aerosol canister.

The actuator is in the form of a hand held device which has a nozzle which can be inserted into the patient's mouth to receive the medicament. The medicament is delivered from an aerosol canister containing a propellant and the particular medicament or drug formulation. The propellant acts to force the drug out of the canister upon actuation of the device.

Actuation of the device is commonly achieved by compression of the stem on an end of the canister which opens a valve and releases a metered dose of medicament into the actuator and on out through the nozzle for inhalation by the patient.

The manufacturing tolerances involved in MDI devices are tight. For example, in order to ensure reliable operation of the actuation valve the movement and alignment of the canister has to be carefully controlled to prevent damage to the valve and/or involuntary actuation release of medicament from the pressurised canister. Typically the depression of any metered dose valve stem by 3 mm or more will cause the device to actuate.

Also, the channel in the actuator into which the stem of the canister is inserted and located is tightly located around the stem to prevent medicament escaping back towards the canister main body and away from the actuator. This valve stem/actuator channel fit requires a ‘push’ force to insert the stem into the actuator. If the push force is too high during the assembly step, the stem will depress and actuate a metered dose of medicament from the pressurised canister.

As stated above this, and other technical requirements, are achieved by tight tolerances on the geometry of the actuator and canister sub-assemblies.

In order to be able to deliver MDI delivery devices at low cost high speed manufacturing, filling and packaging is required where each step has to be carefully controlled to avoid accidental release of medicament and/or damage to the pressured canisters valve stem or actuator.

Furthermore the inventors have established that even a small pre-delivery release of medicament into the actuator passages and nozzle can cause blocking of the MDI. This is because the timeline between manufacture and use may be a number of months or years and the medicament is prone to hardening in the actuator nozzle when exposed to atmosphere over time. This renders the product unusable after delivery to a patient unless the actuator nozzle is cleaned.

A primary step in the manufacturing process is the insertion of the filled and pressurised canister into an actuator ready for packaging and delivery to a patient.

Achieving precise alignment and location of canisters into actuators has conventionally been done with a spring clutch mechanism to prevent the accidental actuation during assembly problems as discussed above. However, the inventors have established an apparatus and method that allows the desired accuracy to be achieved whilst avoiding risk of release of medicament into the manufacturing environment whilst simultaneously allowing extremely high manufacturing rates to be realised.

### SUMMARY

According to a first aspect of an invention disclosed herein there is provided an apparatus for inserting a canister into an inhaler actuator device, said apparatus comprising an inhaler actuator device support member at a first end of said apparatus and an insertion device at a second end adapted to cause a canister to move relative to the actuator device and to enter an open end of said actuator device, wherein the apparatus further comprises a force sensor adapted to measure a reaction force between the canister and actuator device as the canister moves relative to the actuator device.

The canister is secured to the inhaler activation device by locating a stem of the canister into a corresponding stem receiving channel formed in what is termed the ‘stem block’ of the inhaler device. A light press fit secures the outer wall of the canister stem to the inner wall of the channel to locate and hold the canister within the device.

Simultaneously measuring the push force of a canister being inserted into an actuator stem block provides a number of advantages.

For example, a reaction or resistive force is generated as the canister stem is pushed into the channel and the inventors have established that measuring the reaction force advantageously allows for the identification of canisters that have experienced excessive insertion force, and allows for their automatic rejection.

Canisters are designed to have a particular activation force i.e. the force which is required to be applied to the stem to cause the stem to depress thereby causing the valve to operate and release a dose of medicament. If the reaction force is higher than a predetermined force then this may indicate that there is a problem with the assembly step. One cause is where the tip of the valve step catches on the outside edge of the stem block bore and this results in immediate depression of the valve stem and accidental actuation. Additionally, or alternatively, if there is damage to the stem it will jam in the receiving channel and relative movement of canister and actuator will cause the stem to depress, again with an accidental actuation.

Thus, the invention also allows for damaged or defective canisters (valve damage or stem damage) to be identified as part of the existing step of assembling the actuator and canister i.e. an integrated product quality control step is realised without the need for an additional check. This facilitates high speed and high volume manufacturing.

A further advantage is that accidental release of medicament can be avoided. As stated above each canister has an actuation force; a force at or above the actuation force causes the stem to depress and medicament is accidentally released. During assembly if the canister is forced into position too quickly and/or with too great a force the canister may accidentally be activated thereby releasing medicament. This accidental medicament release presents a number of problems including:
the medicament may harden and block the nozzle in storage
the assembly facility and workspace is contaminated with medicament
exposure of operatives to released medicament

By measuring the reaction force and comparing it to the activation force for the given canister it is possible to establish if any medicament has been released or not. Furthermore, it is possible to control the movement of a canister so as to proactively prevent accidental release/actuation. Still further defective canisters can be accurately and quickly identified.

The determination described above may be achieved using a suitable controller and force measuring device. Such a device may for example be adapted to receive an input from the force sensor (such as a load cell) and to compare the measured reaction force against a predetermined reaction force limit for the canister/actuator device combination.

The controller or computer may for example be adapted to output a signal and/or record or output data indicating that a predetermined reaction force has been met or exceeded. If the force is exceeded the canister will be automatically rejected from the line. This thereby allows an operator to be alerted and allows a record to be stored of canisters which are either defective or have been activated accidentally at the assembly stage.

Each canister valve design has its own standard actuation force and so the controller may be provided with a plurality of predetermined reaction force limits corresponding to different canister/actuator combinations. The controller may further be provided with a menu selector permitting a user to conveniently select from the plurality of predetermined reaction force limits. In another arrangement the controller may be arranged to identify the canister type and automatically select the appropriate force parameters.

For example, a first of said plurality of reaction force limits may be approximately 20 Newtons and a second of said plurality of reaction forces may be approximately 30 Newtons.

The controller may further be adapted to actively control the movement of the canister with respect to the actuator using a feedback control arrangement. Thus, the controller may be arranged to output a signal to prevent movement of the insertion device if a predetermined reaction force is reached or exceeded.

The apparatus may be configured such that the canister is only permitted to move by a predetermined maximum displacement from a datum position. Thus, a distal end of a stem of the canister can be located within a stem receiving channel of the actuator device.

The force sensor may be any suitable sensor which can measure or determine the force which is being applied to the canister stem by virtue of its contact with the stem block. This may for example be a load cell manufactured by Kistler Instrumente AG.

Advantageously the force sensor may be located between the insertion device and a portion of the apparatus arranged to apply a moving force to the canister. Thus the forces being applied through the assembly apparatus can be accurately determined by placing the sensor ‘in-line’ with the movement arrangement.

The controller may advantageously be arranged to continuously process the measured reaction force with respect to the predetermined reaction force limit and to control the movement of the insertion device to maintain the measured reaction force below the reaction force limit.

The insertion device which moves the canister into the actuator may be any suitable device but may advantageously be a pneumatically driven cylinder. The controlled may be arranged to interface with the cylinder's own control arrangement (as mentioned above) to control the displacement of the cylinder and thereby the location and speed of the canister with respect to the actuator. Thus, a feedback control can be realised and the force applied to the canister stem can be controlled.

Viewed from another aspect there is provided an aerosol inhaler assembly apparatus comprising a first portion arranged to support an inhaler actuation device and a second portion arranged to support an aerosol canister, said apparatus being arranged to move the aerosol canister into an assembled position within the inhaler actuation device and wherein as the aerosol canister is moved a reaction force between the actuation device and the canister is measured.

Viewed from yet another aspect there is provided a method of inserting a canister into a canister actuation device comprising the steps of causing a canister to move into an open end of a canister actuation device and simultaneously measuring a reaction force between said canister and said canister actuation device.

### BRIEF DESCRIPTION OF THE ACCOMPANYING FIGURES

Specific embodiments of the present invention will be described by way of example only and with reference to the accompanying figures in which:—

FIG. 1 shows two sub-components of a simple metered dose inhaler;

FIG. 2 shows a cross-section of an actuator;

FIG. 3 shows an end view of an actuator;

FIG. 4 shows a valve stem and stem block in detail;

FIG. 5 shows an illustrative ‘damaged’ valve stem;

FIG. 6 is a schematic of the assembly machine; and

FIG. 7 is a displacement force diagram.

While the invention is susceptible to various modifications and alternative forms, specific embodiments are shown by way of example in the drawings and are herein described in detail. It should be understood, however, that the drawings and detailed description of the specific embodiments are not intended to limit the invention to the particular forms disclosed. On the contrary, the invention covers all modifications, equivalents and alternatives falling within the spirit and the scope of the present invention as defined by the appended claims.

### DETAILED DESCRIPTION

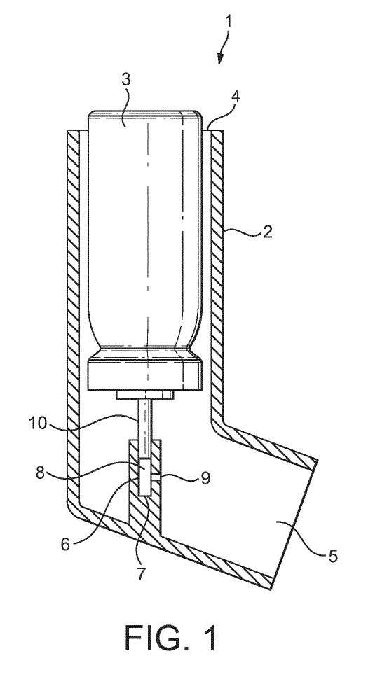

FIG. 1 shows two sub-components of a metered dose inhaler in partial cross-section.

The metered dose inhaler 1 is made up of 2 fundamental subcomponents, an actuator device 2 and an aerosol canister 3.

The actuator 2 has a cylindrical opening 4 to receive the stem of a cylindrical canister 3 at one end and an output nozzle mouthpiece 5 at the other which is placed into the mouth of a user to inhale the medicament. The actuator is configured to activate the canister by means of a channel 6 formed in a stem block 7. The channel 6 is aligned such that an opening 8 can receive a stem of a canister (described in more detail below).

The channel 6 is also in fluid communication with a medicament dispersing diffuser 9 which receives medicament from the channel and diffuses it into the nozzle 5.

The canister 3 comprises a cylindrical body containing a propellant and medicament and metered dose valve with a projecting valve stem 10. Aerosol containers or canisters of this type are very well known in the art and will not be described in detail save as to say that axial movement or depression of the valve stem 10 causes a metered dose of medicament entrained in the propellant to be expelled from an end of the valve stem.

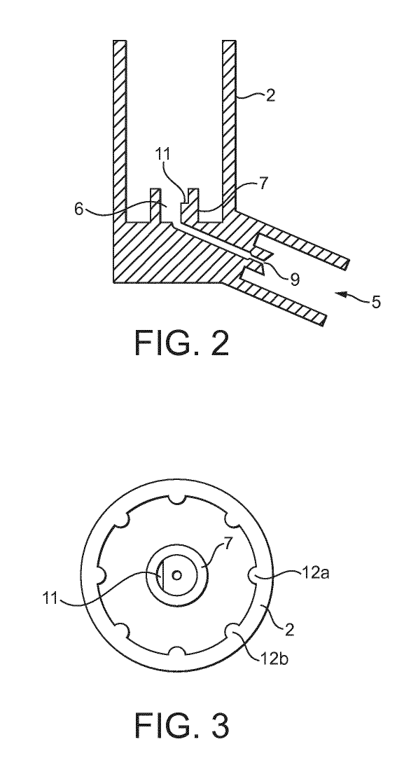

FIG. 2 shows a cross-section of another actuator 2 with like reference numerals referring to like features. As shown in FIG. 2 (and shown in more detail in FIG. 4) the stem block 7 is provided with a projection 11 on an inner surface of the channel 6 against which a valve stem engages. The projection 11 provides an abutment preventing downward movement of the valve stem and causing the relative movement of the canister body and valve stem to cause the actuation to occur.

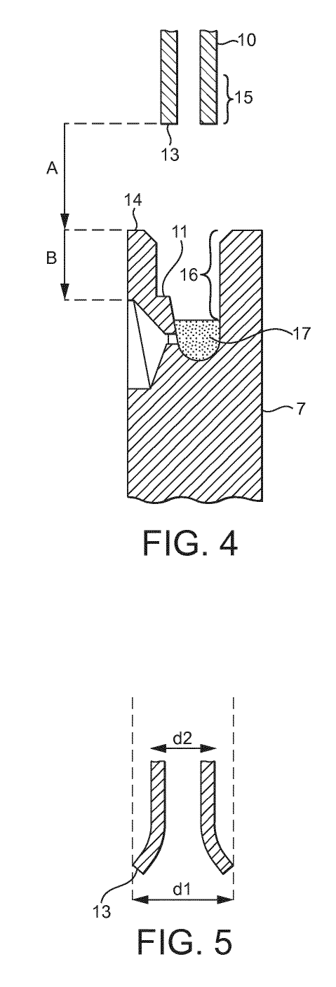

FIG. 3 is an end view of the actuator viewed into the generally cylindrical end which receives the canister. The stem block 7 and projection 11 can be seen in the end view of FIG. 3. FIG. 3 also shows optional support ribs 12a, 12b . . . which circumferentially support the canister once in-situ.

The inhaler assembly is achieved before delivery to a patient by inserting a full canister into the actuator body such that the valve stem is located within the channel 6. The valve stem may extend all the way into the channel in abutment with the projection 11 such that it is ready to operate i.e. a user pressing the end of the canister (the upper part as seen in FIG. 1) causes the valve stem to be compressed against the projection and medicament is released.

The canister valve stem is secured within the actuator stem block by a light press fit between the inner surface of the channel 6 and the outer surface of the valve stem 10. The ribs provide radial support for the canister and additionally assist with aligning the canister coaxially with respect to the actuator during assembly. Importantly the valve stem has to be aligned with the stem block channel as the canister is inserted into the actuator as will be discussed below.

Turning to FIG. 4 there is shown an expanded view of the stem block 7 and valve stem 10. As shown the channel 6 has a projection 11 arranged to abut with the distal end 13 of the valve stem 10 on insertion of the stem into the stem block.

As described above, one of the problems that the inventors have identified (and solved) is that assembly of the canister into the actuator can result in accidental activation of the canister valve. This may be caused by a number of reasons.

One reason for accidental activation of the canister is damage to the valve stem. FIG. 5 illustrates an example of a splayed (expanded) end of the valve stem 10 where the outer diameter d1 is greater than the normal diameter d2. Because the valve stem channel 6 is adapted to closely match the diameter of the given valve stem (so as to provide the necessary interference fit to secure the canister in the actuator) any damage such as that shown in FIG. 5 will cause the end of the valve stem 13 to abut the upper surface 14 of the stem block. This creates a reaction force which quickly exceeds the activation force for the canister causing the stem to depress and medicament to be accidentally released during the assembly process.

It will be recognised that corresponding damage to the stem block in the actuator might equally cause accidental activation of the canister.

Returning to FIG. 4 the canister is assembled by causing the canister to first move along distance A such that the distal end 13 of the valve stem 10 aligns with the stem block. Next the canister is moved along distance B to slide the valve stem into the stem block. It is here where further accidental activation can occur.

As the stem moves into the block the outer surfaces 15 of the valve stem engage with the inner surfaces 16 of the stem block. A reaction force is generated by virtue of the friction (both dynamic and static) against the force which is being applied to cause the movement of the canister.

As one example accidental activation can occur if this reaction force is permitted to exceed the actuation force of the given canister. As an example the metered dose valve of a canister manufactured by the 3M Company has an actuation force of 30 Newtons.

If accidental actuation occurs, a dose of medicament 17 will be discharged into the channel. In the absence of inhalation by a user it will remain in the channel causing the channel to become blocked.

In any of these circumstances the actuator or canister in question must be discarded automatically by the control system on the line.

Thus, measurement of the reaction forces being generated as the canister and actuator are assembled can not only be used to identify defective canisters or defective actuators but also to determine if an accidental actuation has occurred that could cause a blockage of the actuator as described above.

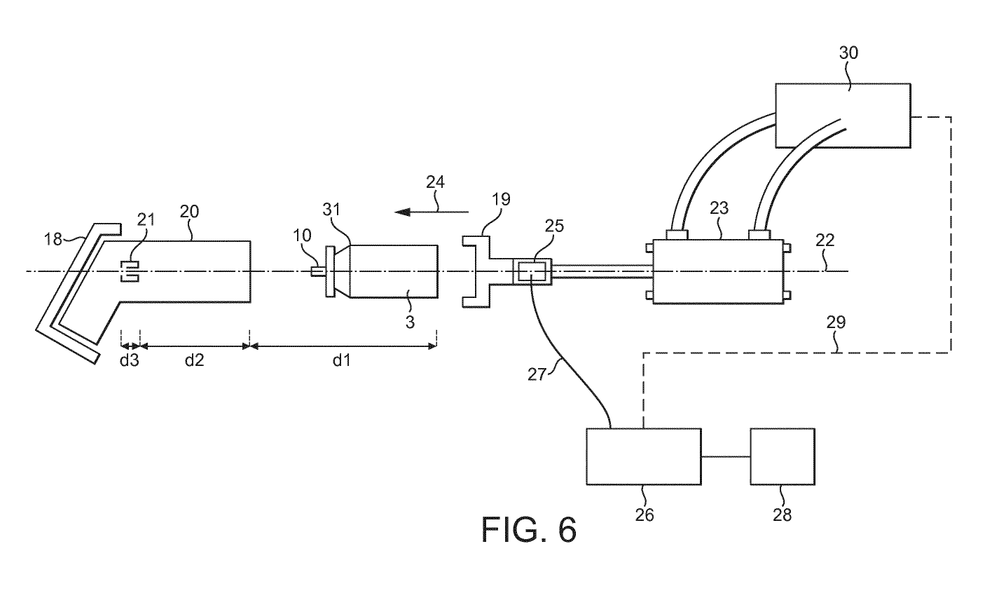

The assembly apparatus and method will now be described with reference to FIG. 6 which is a schematic showing the general arrangement and sub-components of the assembly machine.

The assembly machine comprises an actuator support portion 18 and an opposing canister support portion 19. The actuator support portion is arranged to support an actuator 20 such that a stem block 21 is aligned with a longitudinal axis 22 of the machine. It will be recognised that the actuator may be supported in a range of different ways. The important feature of the actuator support being that it aligns the stem block with the axis 22.

The canister support portion 19 is adapted to support and hold the canister and further to be coupled to a linear actuator 23. The canister support portion 19 is also arranged such that the valve stem 10 of the canister is aligned with the axis 22 such that movement of the canister with respect to the actuator maintains alignment of the stem block 21 and valve stem 10.

The canister support portion 19 is connected on an opposing side to a pneumatically driven linear actuator 23 which, when operated, causes the canister support portion 19 to move along the axis of the machine 22 in the direction 24. Thus, the canister can be inserted into the actuator.

A force sensor in the form of a Kistler load cell 25 is located between the pneumatic linear actuator 23 and the canister support portion 19. Any reaction force generated along the axis of the machine (for example by abutment of a damaged valve stem against the stem block 21) which causes a load to be applied to the sensor 25. The load sensor is provided with a control arrangement 26 which received output signals from the sensor along control lines 27.

The control arrangement 26 is provided with a plurality of predetermined reaction force limits matching the activation forces of various canister and actuator combinations. An operator is able to interface with the controller via interface 28 to select the correct reaction force limit for the current canister and actuator combination.

The controller may be optionally provided with feedback control lines 29 which communicate with the control arrangement 30 for the pneumatic linear actuator 23. The control arrangement 30 is arranged to cause the canister supporting portion to reciprocate between a loading position where a new canister and actuator can be laid onto the machine and an assembled position where the canister is moved into the actuator and the valve stem at least part way into a channel in the stem block 21.

The control line 29 allows the controller 26 to optionally control the movement of the linear actuator so as to ensure that the reaction force remains below a predetermined limit, for example the activation force for the given canister less a tolerance.

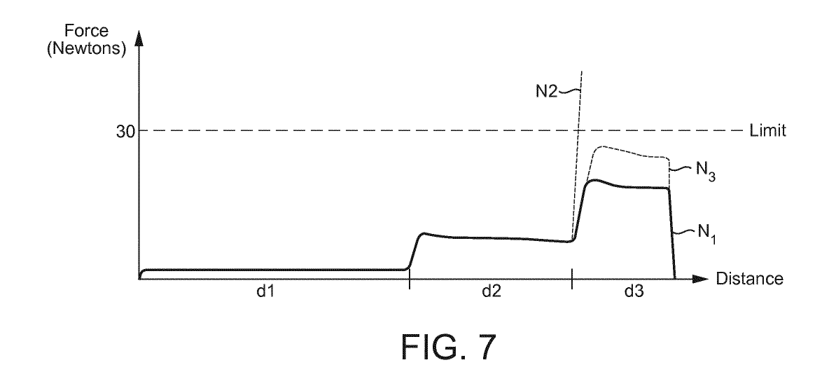

The operation of the machine will now be described with reference to FIG. 6 and FIG. 7 which is a displacement force diagram.

First, a canister and actuator pair is inserted into their respective support portions of the machine. The control arrangement is activated and the pneumatic linear actuator causes the canister to move along the axis 22 and through the distances d₁, d₂ and d₃ shown in both FIG. 6 and FIG. 7.

FIG. 7 is a graph showing force (N) versus distances d₁, d₂ and d₃ along the machine.
d₁ corresponds to the distance between the linear actuator's loading position
d₂ corresponds to the distance of movement of canister into the actuator; and
d₃ corresponds to the distance of movement into the stem block.

During the movement of the canister the control arrangement continuously receives signals from the load cell 25 which are converted into reaction force data which is continuously compared against the activation force setting which has been selected by the user via the interface 28.

FIG. 7 shows how the forces measured by the load cell change as the canister is moved into an assembled position in the actuator.

As the canister moves through a first distance d₁ after a small initial rise caused by overcoming static friction the reaction force is low because there is no resistance to movement of the canister.

At distance d₂ the canister shoulder 31 engages with the ribs shown in FIG. 3 and a small increase in force is seen owing to the slight resistance to movement as the canister outer wall slides along the ribs.

The three examples below represent three different scenarios illustrated by lines N1, N2 and N3 in FIG. 7.

Line N1 is a Non-Defective Canister i.e. a Canister with an Undamaged Valve Stem.

As the valve stem enters the stem block the outer surface engages tightly with the inner surface of the block to cause the interference fit. An initial increase in force is seen which then reduces slightly and finally falls to zero when movement of the canister support portion stops. In this example the canister has been accurately inserted into the actuator. The canister support portion can be retracted and the assembled canister and actuator removed for packaging. The reaction for limit has not been exceeded.

### Line N2 Illustrates the Same Graph for a Damaged Valve Stem.

As the valve stem approaches the stem block the damaged end surface (reference 13 in FIG. 4) abuts with the end face 14 of the stem block. This causes an immediate and large increase in reaction force as shown by line N2 at distance d2. Here the reaction force exceeds the reaction force limit shown in FIG. 7 which is detected by the force sensor 25 and control arrangement 26. Here the operator is alerted that the force has been exceeded indicating that the canister is likely to have been activated accidentally. This may be by any suitable signal such as an audible or visual alarm. The controller may additionally be arranged to cause the canister support portion to retract in combination with an alert of a defective canister.

### Line N3 Illustrates an Alternative Feedback Control Arrangement.

Line N3 represents a situation where the valve stem has a minor defect in the geometry of the valve stem. Here, at distance d3 a damaged outer portion of the valve stem engages and abuts partially with the end of the stem block. In this feedback arrangement the force sensors detects the increase in reaction force which approaches the activation force limit. The controller is arranged to slow down the movement of the pneumatic actuator to reduce the reaction force generated (as shown by line N3 over distance d3). The valve stem slowly slides into the stem body as the defect is deflected by the slower movement of the canister support portion.

Thus, the continuous monitoring of the reaction force allows the controller to proactively control the reaction force being generated preventing accidental activation of the valve and furthermore preventing a defective canister being identified which might actually pass the quality test if it is inserted into the assembly with greater care i.e. at a lower speed and resulting lower force.

The location of the sensor head (such as a sensor head manufactured by Kistler) is generally arranged such that it experiences the direct load as imparted on the canister during the insertion step, typically mounted in line on the drive arm. A Kistler load cell may be advantageously used as it is a recognised robust measurement device, but any load cell from equivalent quality instrumentation suppliers would be interchangeable on the design.

1. An apparatus for inserting a canister into an inhaler actuator device, said apparatus comprising an inhaler actuator device support member at a first end of said apparatus and an insertion device at a second end adapted to cause a canister to move relative to the actuator device and to enter an open end of said actuator device, wherein the apparatus further comprises a force sensor adapted to measure a reaction force between the canister and actuator device as the canister moves relative to the actuator device.

2. An apparatus as claimed in claim 1, further comprising a controller adapted to receive an input from said force sensor and to compare the measured reaction force against a predetermined reaction force limit for the canister/actuator device combination.

3. An apparatus as claimed in claim 2, wherein the controller is adapted to output a signal and/or record or output data indicating that a predetermined reaction force has been met or exceeded.

4. An apparatus as claimed in claim 2, wherein the controller is provided with a plurality of predetermined reaction force limits and is provided with a selector permitting a user to select from the plurality of predetermined reaction force limits.

5. An apparatus as claimed in claim 4, wherein a first of said plurality of reaction force limits is approximately 20 Newtons and a second of said plurality of reaction forces is approximately 30 Newtons.

6. An apparatus as claimed in claim 2, wherein the controller is arranged to output a signal to prevent movement of the insertion device if a predetermined reaction force is reached or exceeded.

7. An apparatus as claimed in claim 1, wherein the insertion device is controlled such that the canister is moved into the canister by a predetermined distance.

8. An apparatus as claimed in claim 7, wherein the predetermined distance is such that a distal end of a stem of a metered dose valve of the canister is located within a stem receiving channel in the stem block of the actuator device.

9. An apparatus as claimed in claim 1, wherein the force sensor in located between the insertion device and a portion of the apparatus arranged to apply a moving force to the canister.

10. An apparatus as claimed in claim 9, wherein the force sensor is a piezoelectric force sensor.

11. An apparatus as claimed in claim 2, wherein the controller is arranged to continuously process the measured reaction force with respect to the predetermined reaction force limit and to control the movement of the insertion device to maintain the measured reaction force below the predetermined reaction force limit.

12. An apparatus as claimed in claim 1, wherein the insertion device is a pneumatically driven cylinder.

13. An apparatus as claimed in claim 1, wherein the insertion device is operable to move the canister at different speeds with respect to the actuator device.

14. An apparatus as claimed in claim 13, wherein the insertion device operates at a first speed to bring the canister towards the actuator device and a second speed as a stem of the canister moves into a stem receiving channel of the actuator device.

15. An apparatus as claimed in claim 1, wherein the canister is a metered dose inhaler canister and the actuator device is a metered dose inhaler actuator device.

16. An aerosol inhaler assembly apparatus comprising a first portion arranged to support an inhaler actuation device and a second portion arranged to support an aerosol canister, said apparatus being arranged to move the aerosol canister into an assembled position within the inhaler actuation device and wherein as the aerosol canister is moved a reaction force between the actuation device and the canister is measured.

17. A method of inserting a canister into a canister actuation device comprising:
causing a canister to move into an open end of a canister actuation device; and
simultaneously measuring a reaction force between said canister and said canister actuation device.

18. A method as claimed in claim 17, further comprising:
comparing the measured reaction force against a predetermined reaction force limit for the canister/canister actuation device combination.

19. A method as claimed in claim 18, further comprising:
a controller, wherein the controller at least one of outputs a signal and/or records or outputs data indicating that the predetermined reaction force has been met or exceeded.

20. A method as claimed in claim 19, wherein the controller is arranged to output a signal to prevent movement of the canister if the predetermined reaction force is reached or exceeded.

21. A method as claimed in claim 18 wherein the canister is automatically rejected if the predetermined reaction force is reached or exceeded.

22. A method as claimed in claim 18, wherein the canister actuation device is automatically rejected if the reaction force reaches or exceeds an actuation force of the canister and/or the predetermined reaction force limit for the canister/canister actuation device combination.

23. A method as claimed in claim 18, wherein the controller is arranged to continuously process the measured reaction force with respect to the predetermined reaction force limit and to control the movement of the canister to maintain the measured reaction force below a reaction force limit.

24-25. (canceled)
